(19) United States
(12) Patent Application Publication
(10) Pub. No.: US 2008/0311063 A1
(43) Pub. Date: Dec. 18, 2008

(54) Method for Imparting Curl to Lashes

(75) Inventors: Arvind N. Shah, Suffern, NY (US); Nancy Felter, West Nyack, NY (US); Tameru Getahoun, Parsippany, NJ (US)

Correspondence Address: AVON PRODUCTS, INC., AVON PLACE, SUFFERN, NY, 10901, US

(73) Assignee: Avon Products, Inc., New York, NY (US)

(21) Appl. No.: 12/092,434

(22) PCT Filed: Dec. 14, 2006
(86) PCT No.: PCT/US06/47772

§ 371 Date: May 2, 2008

Related U.S. Application Data
(60) Provisional application No. 60/755,737, filed on Dec. 30, 2005.

(51) Int. Cl. A61K 8/81; A61Q 1/10; A61Q 5/06

(52) U.S. Cl. 424/70.7; 424/70.11

(57) ABSTRACT

There is a method for imparting curl to eyelashes. The method has the step of applying to the eyelashes an anhydrous liquid composition having the following: a) an organic solvent and b) an alkyl cycloalkylacrylate copolymer soluble or dispersible in the solvent. The organic solvent is present in an amount sufficient to dissolve or disperse the alkyl cycloalkylacrylate copolymer. The alkyl cycloalkylacrylate copolymer is present at about 0.1 wt % to about 90 wt % based on the total weight of the composition.

### CROSS-REFERENCE TO PRIOR APPLICATION

This application claims the benefit of PCT Application Serial No. PCT/US06146595 filed Dec. 6, 2006 which in turn claims priority to U.S. provisional application U.S. Ser. No. 60/755,737 filed Dec. 30, 2005.

### BACKGROUND OF THE INVENTION

1. Field of the Invention

The present invention relates to a method for imparting curl to eyelashes.

2. Description of the Related Ar

Mascara compositions are commonly employed by women to highlight and enhance the appearance of eyelashes. Such enhancements may include the impartation of color or tone, volume, i.e , thickness and length, and curl.

Curling mascara compositions have been formulated in the prior art with combinations of hydrophilic and hydrophobic acrylate polymers, rosin esters, and styrene/olefin block copolymers as film formers. One useful type of hydrophobic water dispersible acrylate polymers used in prior art mascara compositions are the alkyl cycloalkylacrylate copolymers. Alkyl cycloalkylacrylate copolymers have also been disclosed as useful in hair and nail compositions, such as in U.S. Pat. Nos. 6,083,516 and 6,267,951.

The level of eyelash curling that can be attained in the prior art has been limited. The limit of curling achieved has been about 55% according to conventional eyelash measurement techniques.

It would be desirable to have a method for imparting a higher degree of curl to eyelashes than heretofore possible.

### SUMMARY OF THE INVENTION

It is an object of the present invention to provide a method for imparting a high degree of curl to eyelashes.

It is a further object of the present invention to provide a method for imparting a high degree of curl to eyelashes corresponding to a curl of 55% or more, preferably about 65% or more, and most preferably 75% or more.

According to these and other objects of the present invention, there is provided a method for imparting curl to eyelashes. The method has the step of applying to the eyelashes an anhydrous liquid mascara composition having the following, a) an organic solvent and b) an alkyl cycloalkylacrylate copolymer soluble or dispersible in the solvent. The organic solvent is present in an amount sufficient to dissolve or disperse the alkyl cycloalkylacrylate copolymer. The alkyl cycloalkylacrylate copolymer is present at about 0.1 wt % to about 90 wt % based on the total weight of the composition.

### DETAILED DESCRIPTION OF THE INVENTION

It was surprisingly found that there could be a method for imparting a higher degree of curl to eyelashes. It was further surprisingly found that there could be a method for imparting a degree of curl corresponding to a curl of 55% or more, preferably about 65% or more, and most preferably 75% or more.

In the present method, a high degree of curl was obtained by incorporating into a mascara composition a sufficiently high level of a film former is one or more copolymers/polymers (hereinafter referred to as copolymers) having alkyl cycloalkylacrylate monomers. The preferred alkyl cycloalkylacrylate copolymers are based on isomeric monomers having the following two formulas A and B:

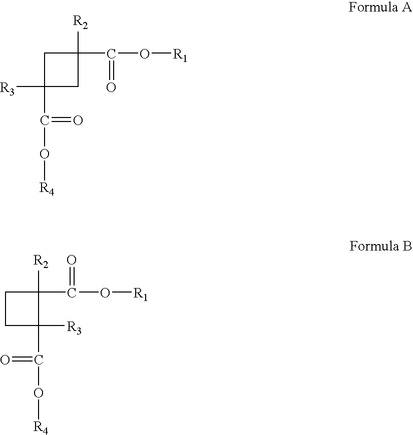

wherein R₁ and R₄ are each hydrogen in the monomeric units but may also represent a terminal group in the final polymer, and R₂ and R₃ are each, independently, hydrogen, methyl, or a C₁₄ to C₃₆ alkyl group, with at least one of R₂ and R₃ being a C14 to C₃₆ alkyl group. Such alkyl cycloalkylacrylate copolymers are also known as alkyl bicycloalkylacrylate copolymers.

The preferred alkyl cycloalkylacrylate copolymers are soluble in hydrocarbons, such as isododecane, but are substantially insoluble in water. Thus, the film-forming effect of the copolymers is suitable for use in an anhydrous system.

Preferably, the alkyl cycloalkylacrylate copolymer is a cycloalkyl methacrylate copolymer having C₁₄ to C₃₆ alkyl units. A preferred cycloalkyl methacrylate copolymer is a bicycloalkyl methacrylate copolymer manufactured by Phoenix Chemicals, Inc., Sommerville, N.J. and sold under the tradename Giovarez AC-5099 ML.

The one or more alkyl cycloalkylacrylate copolymers are present in an amount sufficient to impart a high degree of curl to eyelashes. The alkyl cycloalkylacrylate copolymers are present in an amount from about 0.1 wt % to about 90 wt % based on the total weight of the composition. More preferably, the alkyl cycloalkylacrylate copolymers are present from about 0.5 wt % to about 50 wt %. Most preferably, the alkyl cycloalkylacrylate copolymers are present from about 2 wt % to about 35 wt %.

The mascara composition may also have a second film former in the form of one or more esters containing acids derived from rosin. The preferred esters of the present invention are glyceryl rosinate, pentaerythrityl rosinate, silicone rosinate, and mixtures thereof. Esters of rosin acids are substantially insoluble in water. This insolubility enhances the film-forming effect of the ester.

The esters of rosin acids are preferably present in an amount about 0.10 wt % to about 20 wt % based on the total weight of the composition. More preferably, the esters of rosin acids are present in an amount about 2 wt % to about 10 wt %.

The mascara composition includes one or more cosmetically acceptable organic solvents. The organic solvents in the composition act as carriers for the other ingredients and rapidly and evenly dissolve or disperse the ingredients. The organic solvents may be volatile, non-volatile, or a combination thereof. Volatile organic solvents are preferred since they evaporate relatively quickly and, thus, provide quick setting for the mascara composition. Useful organic solvents include C₆ to C₂₀ hydrocarbons, particularly the isoparaffins. Isoparaffins are disclosed, for example, in U.S. Pat. Nos. 3,439,088 and 3,818,105, both of which are incorporated herein by reference. Preferred isoparaffins are isododecane and isohexadecane. Other useful organic solvents include esters, ethers, and acids derived from fatty acids, as well as lower alcohols and polyhydric alcohols. Other useful solvents include butyl acetate and ethyl acetate. Silicone oils, particularly volatile linear and silicones, are also useful.

The one or more organic solvents are present in an amount sufficient to dissolve or disperse the alkyl cycloalkylacrylate copolymer. Preferably, the organic solvents are present in an amount about 0.1 wt % to about 90 wt % based on the total weight of the composition. More preferably, the organic solvents are present in an amount about 0.5 wt % to about 40 wt %. Most preferably, the organic solvents are present in an amount about 2 wt % to about 30 wt % of the total weight of the composition.

The mascara composition is anhydrous. The term “anhydrous” means water is present in an amount less than 3 wt % and most preferably less than 1 wt % water. The mascara composition takes a liquid or fluid form. The composition typically exhibits the physical consistency of a lotion or cream. The physical consistency is such that the composition can be easily applied to the eyelashes with a mascara brush or similar applicator.

The mascara composition optionally has one or more natural or synthetic waxes therein. The waxes may be hard or soft. Suitable waxes include, but are not limited to, abies alba leaf wax, acacia dealbata flower wax, acacia decurrens flower wax, acacia farnesiana flower wax, beeswax, carnauba acid wax, ceresin, cetyl esters, cistus ladaniferus flower wax, citrus aurantium amara (bitter orange) flower wax, citrus aurantium dulica (orange) flower wax, citrus aurantium dulcis (orange) peel wax, copernicia certifera (candelilla) wax, cytisus scoparius flower wax, edipta prostrata wax, ericerus pela wax, euphorbia cerifera (candelilla) wax, helichrysum angustifolium wax, hydrogenated Japan wax, hydrogenated microcrystalline wax, hydrogenated rica bran wax, hydrolyzed beeswax, hydrolyzed candelilla wax, hydrolyzed carnauba wax, iris florentina flower wax, jasminum gradiflorum (jasmine) flower wax, jasminum officinale (jasmine) flower wax, jasminum sambac (jasmine) flower wax, jojoba esters, lanolin wax, lavandula angustifolia (lavander) flower wax, lowsonia lnermis wax, microcrystalline wax, mink wax, montan acid wax, montan wax, myrica cerifera (bayberry) fruit wax, ocimum tanuiflorum oil, oryza sativa (rice) bran wax, ouricury wax, oxidized beeswax, oxidized microcrystalline wax, ozakerite, palm kernel wax, paraffin, peg-6 beeswax, peg-8 beeswax, peg-12 beeswax, peg-20 beeswax, peg-12 carnauba, persia gratissima (avocado) wax, pistacia tentiscus leaf wax, polianthes tuberosa flower wax, potassium oxidized microcrystalline wax, pyrus malus (apple) peel wax, rhus succedanea fruit wax, ribes nigrum (black currnt) bud wax, rosa centifolia flower wax: rosa hybrid flower wax, rosa mutiflora flower wax, salvia sclarea (clary) flower/leaf/stem wax, shellac wax, stipa tenacissima wax, sulfurized jojoba oil, synthetic beeswax, synthetic candelilia wax, synthetic carnauba, synthetic Japan wax, synthetic jojoba oil, synthetic wax, and vitis vinifera (grape) leaf wax.

The mascara composition preferably has one or more pigments, pearlescents, and/or colorants therein. Useful pigments and/or colorants include any known in the cosmetic art. Coatings and surface treatments on pigments may also enhance the shine or gloss exhibited by mascara compositions. Examples of useful pigments include titanium dioxide, zinc oxide, iron oxide, chromium oxide, ferric blue, and mica. Organic pigments include barium, strontium, calcium or aluminum lakes, ultramarines, and certified D&C #2. Colorants include D&C Green #3, D&C Yellow #5, and D&C Blue #1, Pigments and/or colorants may be coated or surface treated with one or more compatibilizers to aid in dispersion in either or both of the aqueous or wax phases. When silicone vehicles are used, pigments and/or colorants surface treated with dimethicone copolyol are preferred. Pearlescent agents are understood as meaning iridescent particles, in particular, particles produced by certain molluscs in their shell or synthesized pearlescent particles. Pearlescent agents particularly serve to modify the texture of the composition.

Pigments can be present in the mascara composition in an amount of 0.001 wt % to about 25 wt % and preferably in an amount of about 1 to about 15 wt % based on the total weight of the composition.

The mascara composition optionally has a plurality of discrete fibers therein. Fibers add volume and length to eyelashes. Fibers may be derived from synthetic, natural, or mineral materials. Synthetic fibers, such as nylon and polymethylmethacrylate, are preferred. Fibers typically have a length of about 0.001 mm to about 10 mm, more typically about 0.1 mm to about 5 mm, and most typically about 0.5 mm to about 2.5 mm. Fibers may be present in the composition at from about 0.01 to about 20 wt %, preferably about 0.1 to 10 wt % , and most preferably about 0.1 to about 5 wt %.

The mascara composition may further have any ingredient conventionally used in the cosmetic field, in particular those used in the manufacture of mascara and eyelash products. The amounts of these various ingredients are those conventionally used in the cosmetic field to achieve their intended purpose, and range typically from about 0.01 wt % to about 20 wt % by weight of the composition. The nature of these ingredients and their amounts must be compatible with the production of stable mascara compositions useful in the method of the present invention.

The mascara composition may also include one or more of the following optional ingredients: amino acids, antioxidants, emollients, emulsifiers, eyelash protectants, eyelashes penetration enhancers, fillers, moisturizers, preservatives, surfactants, thickeners, vitamins, waxes, and combinations thereof.

The mascara composition may be applied to the eyelashes as often as needed to maintain the desired curl. The composition is typically applied to the upper side of the natural hairs with an outward, extending motion (in the general direction of the extension of the hairs). Application results in a film coating on at least a portion of the eyelashes. The composition then dries through evaporation of solvent and any other volatiles. Periodic re-application will be necessary in the normal course as the film coating wears off. Although not limited per se, the curl will typically be maintained for about 4 to about 72 hour.

In the present method, application of the mascara composition provides a degree of curl to eyelashes corresponding a curl of 55% or more, preferably about 65% or more, more preferably about 80% or more, and most preferably about 95% or more obtained by the film former shrinkage test. Degree of curl is determined by measuring degree of curl observed in artificial eyelashes after application of the mascara composition. Bundles of artificial lashes are glued to the periphery of a holding cylinder. The mascara composition is applied to the lashes for fourteen strokes with a mascara brush. After at least ten minutes of drying time, an image, such as photographic or digital image, is taken from a side view. Percent (%) Curl=[(R−L)/R]×100%. Length measurement (L) is determined by measuring from the root of a bundle and the tip of a lash in sharp focus. Curve length measurement (R) is determined by measuring the length of the same lash hair from the root to the tip in small steps. If the lash hair is not clearly distinguishable from the rest of the bundle, the center of the bundle is traced.

For purposes of pre-characterization and pre-qualification of prospective film formers another test referred to as the film former shrinkage test may be employed. Unlike the aforementioned test employing false eyelashes, the film former shrinkage test employs fabric swatches. The film former shrinkage test is described in detail below.

The following are examples of the present invention. Unless otherwise indicated, all percentages or parts are by weight.

### EXAMPLES

### Examples 1 and 2 and Comparative Examples 1 and 2 and Control #1

Anhydrous compositions having a film former(s) and an organic solvent were prepared and tested for curling via the film former shrinkage test.

In the compositions of the invention, Examples 1 and 2, an alkyl cycloalkylacrylates copolymer was employed at 30 wt % as the film former based on the total weight of the composition. Examples 1 and 2 both used isododecane (IDD) as the solvent. In the compositions of Comparative Examples 1 and 2, trimethylsiloxy silicate was used as the film former and cyclomethicone pentamer (available as KF 7312J from Shin Etsu, a 50/50 mixture of the polymer and the solvent) was used as the solvent. In the composition of Control 1 glycerol rosinate was used as the film former and IDD was used as the solvent.

In the film former shrinkage test degree of curling is quantified. The test compositions have 30 wt % of polymer (based on the total weight of the composition) in an amount of organic solvent sufficient to dissolve or disperse it. Wool fabric swatches of 11.43 cm by 7.62 cm dimension are soaked with test compositions for 30 seconds, hung up on the short side, and line-dried in a control room overnight at 40% relative humidity and a temperature of 24″ C. After treatment, the dried swatches are placed on a flat surface, and the long side with the least curl is held to the surface by a weight. The swatch is placed so the free long side with the greater curl can project up away from the flat surface. The length of the free long side of the swatch that lies flat in contact with the surface is measured (the layflat length). No shrinkage would have a measured layflat length of 11.43 cm. High shrinkage would have a measured layflat length of near zero. Percent shrinkage is calculated as follows:

[(Swatch length−Avg. Layflat length)/Swatch length]×100=% shrinkage

or

[(11.43−Avg. Layflat length)/11.43]×100=% shrinkage

After treatment, the swatches were examined and measured for layflat length vs. curve length (swatch length). Ten tests were carried out for each composition.

The results are set forth in Tables 1 to 5 below.

The anhydrous compositions of Examples 1 and 2 yielded significantly greater average curve lengths than the compositions of Comparative Examples 1 and 2 and Control 1. The greater average curve lengths correlates to a greater degree of curl when applied to eyelashes.

TABLE 1
Example 1
(30% alkyl cycloalkylacrylates copolymer¹ in IDD²)

| Test # | 1 | 2 | 3 | 4 | 5 | 6 | 7 | 8 | 9 | 10 |
|---|---|---|---|---|---|---|---|---|---|---|
| Layflat | 0.00 | 0.00 | 0.00 | 0.00 | 0.00 | 0.00 | 0.00 | 0.00 | 0.00 | 0.00 |
| length(cm) | | | | | | | | | | |
| Curve | 11.43 | 11.43 | 11.43 | 11.43 | 11.43 | 11.43 | 11.43 | 11.43 | 11.43 | 11.43 |
| Length(cm) | | | | | | | | | | |

Avg. Curve Length - average curve length
STDeV - standard deviation
¹Incorporated as 60% Giovarez AC-5099 ML from Phoenix Chemical which is a 50/50 mixture of cycloalkylmethacrylate polymer in isododecane solvent.
²Q.S. to 100%
Avg. Curve Length 11.43
STDeV 0.0
% Shrinkage 100.00%

TABLE 2
Example 2
(30% alkyl cycloalkylacrylates copolymer¹/glycerol rosinate (50/50) in IDD²)

| Test # | 1 | 2 | 3 | 4 | 5 | 6 | 7 | 8 | 9 | 10 |
|---|---|---|---|---|---|---|---|---|---|---|
| Layflat | 0.00 | 0.50 | 0.00 | 0.00 | 0.00 | 0.00 | 0.00 | 0.00 | 0.00 | 0.00 |
| length(cm) | | | | | | | | | | |
| Curve | 11.43 | 10.93 | 11.43 | 11.43 | 11.43 | 11.43 | 11.43 | 11.43 | 11.43 | 11.43 |
| Length(cm) | | | | | | | | | | |

¹Incorporated as 60% Giovarez AC-5099 ML from Phoenix Chemical, which is a 50/50 mixture of cyloalkylmethacrylate polymer in isododecane solvent.
²Q.S. to 100%
Avg. Curve Length 11.38
STDeV 0.16
% Shrinkage 99.56%

TABLE 3
Comparative Example 1
(30% trimethylsiloxy silicate¹ in cyclomethicone pentamer²)

| Test # | 1 | 2 | 3 | 4 | 5 | 6 | 7 | 8 | 9 | 10 |
|---|---|---|---|---|---|---|---|---|---|---|
| Layflat | 0.00 | 4.00 | 5.00 | 0.00 | 0.50 | 4.00 | 2.00 | 3.00 | 2.00 | 4.00 |
| length(cm) | | | | | | | | | | |
| Curve | 11.43 | 7.43 | 6.43 | 11.43 | 10.93 | 7.43 | 9.43 | 6.43 | 9.43 | 7.43 |
| Length(cm) | | | | | | | | | | |

¹Incorporated as 60% Shin-Etsu KF 7312J, a 50/50 mixture of trimethylsiloxy silicate in cyclomethicone pentamer.
²Q.S. to 100%
Avg. Curve Length 6.98
STDeV 1.63
% Shrinkage 78.57%

TABLE 4
Comparative Example 2
(30% trimethylsiloxy silicate¹ in cyclomethicone pentamer²)

| Test # | 1 | 2 | 3 | 4 | 5 | 6 | 7 | 8 | 9 | 10 |
|---|---|---|---|---|---|---|---|---|---|---|
| Layflat | 3.00 | 6.00 | 2.00 | 0.00 | 0.00 | 0.50 | 2.00 | 0.50 | 1.00 | 1.00 |
| length(cm) | | | | | | | | | | |
| Curve | 8.43 | 5.43 | 9.43 | 11.43 | 11.43 | 10.93 | 9.43 | 10.93 | 10.43 | 10.43 |
| Length(cm) | | | | | | | | | | |

¹Incorporated as 60% Shin-Etsu KF 7312J, a 50/50 mixture of trimethylsiloxy silicate in cyclomethicone pentamer.
²Q.S. to 100%
Avg. Curve Length 9.83
STDeV 1.82
% Shrinkage 86.00%

TABLE 5
Control 1
(30% glycerol rosinate in isododecane)

| Test # | 1 | 2 | 3 | 4 | 5 | 6 | 7 | 8 | 9 | 10 |
|---|---|---|---|---|---|---|---|---|---|---|
| Lay flat | 0.00 | 0.50 | 0.50 | 1.00 | 0.00 | 4.00 | 1.00 | 0.00 | 0.00 | 0.00 |
| length(cm) | | | | | | | | | | |
| Curve | 11.43 | 10.93 | 10.93 | 10.43 | 11.43 | 7.43 | 10.43 | 11.43 | 11.43 | 11.43 |
| Length(cm) | | | | | | | | | | |

* Each fabric swatch was soaked in solution for 30 seconds. Line dried in the humidity (40%) and temperature (24 C.) control room over night.
Avg. Curve Length 10.73
STDeV 1.23
% Shrinkage 93.88%

### Example 3

A mascara composition useful in the method of the present invention was prepared. Ingredients are set forth in Table 6.

TABLE 6

| Ingredient | Wt % | Phase |
|---|---|---|
| Isododecane | 25.60 | A |
| Isohexadecane | 9.60 | A |
| Silica-fumed | 2.50 | B |
| Beeswax | 1.00 | A |
| Glycerol Rosinate (food grade) | 4.00 | A |
| Sorbitan monostearate | 2.50 | A |
| Ozokerite 170-D | 5.00 | A |
| Shellac Wax 100% | 2.00 | A |
| Giovarez AC-5099 ML** | 4.00 | A |
| Polyglycerol Diisostearate | 0.125 | A |
| Propylparaben | 0.20 | A |
| Methylparaben | 0.20 | B |
| Corn Starch | 2.00 | B |
| Sericete - 100% [check spelling] | 3.15 | B |
| Iron Oxide Black - 335198 | 9.00 | C |
| Fiber CB Triangular (NFCB-100-2T)* | 1.00 | D |
| D + C Black No. 2/Gum | 0.50 | C |
| Arabic/AQ/Pres. Dsp. | | |
| Clear Lipstick Base | 29.625 | A |

*from Suppliers KoBo Products INC. INCI Name: Nylon-6, Black-2
**Cycloalkylmethacrylate polymer (50% active in isododecane solvent)

### Directions for Making

Phase A—Weigh out in beaker. Heat to 180 F. Mix with homogenizer. Keep covered.

Phase B—Add to phase A Mix well until uniform.

Phase C—Add colorants to Phase A. Mix well until uniform, Check to see if pigment is dispersed Remove to sweep blade. Cool batch to 120° F.

Phase D—Add to phase A at 120° F. Mix until uniform. Cool to 75° F.

### Example 4, Comparative Example 2, and Control 2

Anhydrous compositions having a film former(s) and an organic solvent were prepared and tested for curling.

In the composition of the invention Example 4, an alkyl cycloalkylacrylates copolymer was employed at 30 wt % as the film former based on the total weight of the composition. Example 4 used isododecane as the solvent. In Comparative Example 3, Shin-Etsu KF 7312J was used as the film former and cyclomethicone pentamer was used as the solvent. In the composition of Control 2, glycerol rosinate was used as the film former and isododecane was used as the solvent.

The tests were carried out as in substantially the same manner as previously. The results are set forth in Tables 7, 8, and 9.

The anhydrous compositions of Example 3 yielded a greater average curve length and percent shrinkage than the compositions of Comparative Example 3 and Control 2. The greater average curve length and percent shrinkage correlate to a greater degree of curl when applied to eyelashes.

TABLE 7
Comparative Example 3
(30% trimethylsiloxy silicate¹ in cyclomethicone pentamer²)

| Test # | 1 | 2 | 3 | 4 | 5 | 6 | 7 | 8 | 9 | 10 |
|---|---|---|---|---|---|---|---|---|---|---|
| Layflat | 0.00 | 4.00 | 5.00 | 0.00 | 0.50 | 4.00 | 2.00 | 3.00 | 2.00 | 4.00 |
| length(cm) | | | | | | | | | | |
| Curve | 11.43 | 7.43 | 6.43 | 11.43 | 10.93 | 7.43 | 9.43 | 8.43 | 9.43 | 7.43 |
| Length(cm) | | | | | | | | | | |

¹Incorporated as 60% Shin-Etsu KF 7312J, a 50/50 mixture of trimethylsiloxy silicate in cyclomethicone pentamer.
²Q.S. to 100%
Avg. Curve Length 8.98
STDeV 1.83
% Shrinkage 78.57%

TABLE 8
Example 3
(30% alkyl cycloalkylacrylates copolymer¹ in IDD²)

| Test # | 1 | 2 | 3 | 4 | 5 | 6 | 7 | 8 | 9 | 10 |
|---|---|---|---|---|---|---|---|---|---|---|
| Layflat | 0.00 | 0.00 | 0.00 | 0.00 | 0.00 | 0.00 | 0.00 | 0.00 | 0.00 | 0.00 |
| length(cm) | | | | | | | | | | |
| Curve | 11.43 | 11.43 | 11.43 | 11.43 | 11.43 | 11.43 | 11.43 | 11.43 | 11.43 | 11.43 |
| Length(cm) | | | | | | | | | | |

¹Incorporated as 60% Giovarez AC-5099 ML from Phoenix Chemical, which is a 50/50 mixture of cycloalkylmethacrylate polymer in isododecane solvent.
²Q.S. to 100%
Avg. Curve Length 11.43
STDeV 0.00
% Shrinkage 100.00%

TABLE 9
Control 2
(30% glycerol rosinate in IDD)

| Test # | 1 | 2 | 3 | 4 | 5 | 6 | 7 | 8 | 9 | 10 |
|---|---|---|---|---|---|---|---|---|---|---|
| Layflat | 0.00 | 0.50 | 0.50 | 1.00 | 0.00 | 4.00 | 1.00 | 0.00 | 0.00 | 0.00 |
| length(cm) | | | | | | | | | | |
| Curve | 11.43 | 10.93 | 10.93 | 10.43 | 11.43 | 7.43 | 10.43 | 11.43 | 11.43 | 11.43 |
| Length(cm) | | | | | | | | | | |

Avg. Curve Length 10.73
STDeV 1.23
% Shrinkage 93.88%

It should be understood that the foregoing description is only illustrative of the present invention. Various alternatives and modifications can be devised by those skilled in the art without departing from scope of the invention. Accordingly, the present invention is intended to embrace all such alternatives, modifications and variances that fall within the scope of the appended claims.

What is claimed is:

1. A method for imparting curl to eyelashes, comprising
applying to the eyelashes an anhydrous liquid composition having
an alkyl cycloalkylacrylate copolymer soluble or dispersible in the solvent and
an organic solvent in an amount sufficient to dissolve or disperse the alkyl cycloalkylacrylate copolymer,
wherein the alkyl cycloalkylacrylate copolymer is present at about 0.1 wt % to about 90 wt % based on the total weight of the composition.

2. The method of claim 1, wherein the alkyl cycloalkylacrylate copolymer is present in an amount about 0.5 to about 40 wt % based on the total weight of the composition.

3. The method of claim 1, wherein the organic solvent is present at about 0.1 wt % to about 90 wt$% based on the total weight of the composition.

4. The method of claim 3, wherein the solvent is selected from the group consisting of isododecane, isohexadecane, and a combination thereof.

5. The method of claim 1, further comprising a pigment, colorant, or pearlescent.

6. The method of claim 1, further comprising a rosin ester,

7. The method of claim 6, wherein the rosin ester is present from about 0.1 wt % to about 20 wt % based on the total weight of the composition.

8. The method of claim 6, wherein the rosin ester is glycerol rosinate.

9. The method of claim 1, further comprising one or more waxes.
